(19) United States
(12) Patent Application Publication
(10) Pub. No.: US 2024/0341910 A1
(43) Pub. Date: Oct. 17, 2024

(54) FIDUCIAL MARKER

(71) Applicant: AUGMEDICS LTD., Yokneam Illit (IL)

(72) Inventors: Stuart Wolf, Yokneam (IL); Nissan Elimelech, Beerotaim (IL)

(21) Appl. No.: 18/631,804

(22) Filed: Apr. 10, 2024

Related U.S. Application Data
(62) Division of application No. 16/524,258, filed on Jul. 29, 2019, now Pat. No. 11,980,506.

(51) Int. Cl. A61B 90/00

(52) U.S. Cl. CPC A61B 90/39; A61B 90/361; A61B 2090/365; A61B 2090/3937; A61B 2090/3966

(57) ABSTRACT

A medical marking device, consisting of a radiotransparent plate having a plurality of radiopaque elements embedded therein in a predefined pattern. The device also has a sigmoid mounting arm having a first end connected to the radiotransparent plate and a second end containing one or more fastening receptacles.

### CROSS-REFERENCE TO RELATED APPLICATIONS

This application is a continuation of U.S. patent application Ser. No. 16/524,258, filed Jul. 29, 2019, which is hereby incorporated by reference herein in its entirety.

### FIELD OF THE INVENTION

The present invention relates generally to a fiducial marker, and specifically to a marker that can be used for registration of multiple frames of reference present in image guided surgery.

### BACKGROUND OF THE INVENTION

In an augmented reality system that is used for image guided surgery the system should track objects used in the surgery, and/or elements of the patient undergoing the surgery. The tracking requires registration of different frames of reference operative during the surgery, including a frame of reference of the patient and a frame of reference of a fluoroscopy facility imaging the patient. The registration typically requires a fiducial marker, and a number of such markers are known. References describing examples of markers are given below.

U.S. Pat. No. 6,314,310 to Ben-Haim et al. describes apparatus for X-ray guided surgery. The apparatus includes a reference element which is placed in contact with the body of a subject, and the element plurality of fiducial marks.

U.S. Pat. No. 7,107,091 to Jutras et al. describes a surgical device that is adapted for use with an image guided surgical system. The device facilitates monitoring inter-dependently mobile bone elements.

U.S. Pat. No. 9,179,984 to Teichman et al. describes a navigation system that includes a multi-configuration tracking array. A plurality of tracking devices can be positioned on the multi-configuration tracking array.

U.S. Pat. No. 9,498,231 to Haider et al. describes computer aided surgery utilizing an on tool tracking system.

U.S. Pat. No. 9,844,413 to Doan et al. describes a monitoring system that tracks the non-visible structure of a body in three dimensions. A tracker obtains image information of an object and instruments in its vicinity, all bearing 3D tracking markers with at least one pattern segment.

U.S. Pat. No. 9,872,733 to Shoham et al. describes a system providing a mechanical guide for drilling the holes for distal screws in intramedullary nailing surgery. The drill guide is automatically positioned by a robot relative to the distal locking nail holes, using data derived from X-ray fluoroscopic images.

U.S. Pat. No. 10,034,713 and U.S. Patent Application 2017/0252109 to Yang et al. describe a system for tracking a position and orientation of a handheld implement. A support member secures one or more markers relative to a longitudinal portion of the handheld implement, and a marker plane containing the markers is orientated at an angle relative to a longitudinal axis of the longitudinal portion.

U.S. Pat. No. 10,080,616 to Wilkinson et al. describes a system that accesses image data of a bone to which a reference marker array is fixed.

U.S. Pat. No. 10,085,709 and U.S. Patent Application 2017/0164919 to Lavallee et al. describe projecting a 3D image on at least part of 2D X-ray images and adjusting projective geometry data of the images, the adjustment comprising registration of the images with the projection of an initial 3D image using an image-to-image registration technique.

U.S. Pat. No. 10,166,079 to McLachlin et al. describes performing intraoperative image registration during a medical procedure. A depth-encoded marker is provided to an object of interest. The marker is imageable by at least two imaging systems, and the marker has asymmetry in at least a depth dimension.

U.S. Pat. No. 10,194,993 to Roger et al. describes a system for aiding surgery on a patient. The system includes a display device and a storage device that stores an image of at least a portion of the anatomy of the patient, including one or more surgical navigation markers positioned on the patient, for display on the display device.

U.S. Patent Application 2011/0004259 to Stallings et al. describes a device for positioning a fiducial marker on an anatomical structure. The device includes a fiducial base and a fixation member. The fiducial base comprises a turn and an extension configured to position the fiducial marker within the field of view of a tracking sensor.

U.S. Patent Application 2011/0098553 to Robbins et al. describes automatic registration of a magnetic resonance (MR) image is carried out in an image guidance system by placing MR visible markers at known positions relative to markers visible in a camera tracking system.

U.S. Patent Application 2015/0150641 to Doan et al. describes a position and orientation tracking system having one or more pattern tags, each tag comprising a plurality of contrasting portions. There is a tracker for obtaining image information about the pattern tags, and a database with geometric information describing patterns on the pattern tags.

U.S. Patent Application 2015/0366620 to Cameron et al. describes a guide for use with an access port for port-based surgery. The guide includes a body positionable over a surgical opening and a grip coupled to the body for removably receiving the access port into the surgical opening.

U.S. Patent Application 2017/0281283 to Siegler et al. describes tracking marker support structures that include one or more fiducial reference markers, where the tracking marker support structures are configured to be removably and securely attached to a skeletal region of a patient.

U.S. Patent Application 2018/0200002 to Kostrzewski describes robotic surgical systems with built-in navigation capability for patient position tracking and surgical instrument guidance during a surgical procedure, without the need for a separate navigation system.

U.S. Patent Application 2018/0318035 to McLachlin et al. describes a reference tie that is to be secured around a portion of a spine during a surgical procedure and that is to be tracked by a surgical navigation system.

U.S. Patent Application 2019/0015163 to Abhari et al. describes how navigational information relative to a site of a medical procedure is determined. The navigational information is then mapped to a common coordinate space, to determine the navigational information relative to a field of view of saved and live optical images of the surgical site.

Documents incorporated by reference in the present patent application are to be considered an integral part of the application except that, to the extent that any terms are defined in these incorporated documents in a manner that conflicts with definitions made explicitly or implicitly in the present specification, only the definitions in the present specification should be considered.

### SUMMARY OF THE INVENTION

An embodiment of the present invention provides a medical marking device, including:
a radiotransparent plate having a plurality of radiopaque elements embedded therein in a predefined pattern; and
a sigmoid mounting arm having a first end connected to the radiotransparent plate and a second end containing one or more fastening receptacles.

Typically the sigmoid mounting arm is radiotransparent.

In a disclosed embodiment the sigmoid mounting arm includes a first curved section connected to a second curved section by a straight section.

In a further disclosed embodiment the sigmoid mounting arm includes a first curved section connected directly to a second curved section.

In a yet further disclosed embodiment the predefined pattern has no axis of symmetry and no plane of symmetry.

There is further provided, according to another embodiment of the present invention, apparatus, including:
a surgical clamp for attachment to a bone of a patient; and
a marking device, which includes
a radiotransparent plate having a plurality of radiopaque elements embedded therein in a predefined pattern; and
a sigmoid mounting arm having a first end fixedly connected to the radiotransparent plate and a second end containing one or more fastening receptacles configured for removable connection of the arm to the surgical clamp.

There is further provided, according to another embodiment of the present invention, a method, including:
embedding a plurality of radiopaque elements, arranged in a predefined pattern, in a radiotransparent plate; and
connecting a first end of a sigmoid mounting arm to the radiotransparent plate, the arm having a second end containing one or more fastening receptacles.

There is further provided, according to another embodiment of the present invention, a method, including:
attaching a surgical clamp to a bone of a patient;
embedding a plurality of radiopaque elements, arranged in a predefined pattern, in a radiotransparent plate;
connecting a first end of a sigmoid mounting arm to the surgical clamp, the arm having a second end fixedly connected to the radiotransparent plate, and having a predetermined mechanical offset between the first end and the second end;
fluoroscopically scanning the radiotransparent plate, the sigmoid mounting arm, and the surgical clamp so as to form a fluoroscopic scan; and
in response to the predetermined mechanical offset and the fluoroscopic scan, determining a spatial transformation between the patient and the surgical clamp so as register a frame of reference of the patient with a frame of reference of the surgical clamp.

In an alternative embodiment, the method includes, in response to registration of the frames of reference, presenting to a professional performing surgery on the patient a stored image of the patient aligned with the patient.

In a further alternative embodiment the predefined pattern and the predetermined mechanical offset are in a one-to-one correspondence.

The present disclosure will be more fully understood from the following detailed description of the embodiments thereof, taken together with the drawings, in which:

### BRIEF DESCRIPTION OF THE DRAWINGS

FIG. 1 is a schematic diagram illustrating a medical marking device as it is used during a preparatory stage of a medical procedure, according to an embodiment of the present invention;

FIG. 2 is a schematic view of the device 10 separated from a clamp, according to an embodiment of the present invention;

FIG. 3 is a schematic exploded view of the device, according to an embodiment of the present invention;

FIG. 4 is a flowchart of steps performed in both a preparatory stage and a subsequent stage of a procedure, according to an embodiment of the present invention; and

FIG. 5 is a schematic illustration of the subsequent stage of the procedure, according to an embodiment of the present invention.

### DETAILED DESCRIPTION OF EMBODIMENTS

### Overview

An embodiment of the present invention provides a fiducial marker that is used to register different frames of reference that are present in image guided surgery on a patient. The image guided surgery may be performed by a medical professional wearing an augmented reality head-mounted display, and in order for the display to operate correctly, images of the patient presented to the professional should align with the actual patient. The registration provided by the fiducial marker ensures the necessary image alignment.

In a disclosed embodiment the marker is connected to a clamp which has been attached, in a preparatory stage of a procedure performed on a patient, to one or more spinous processes of the patient. The marker comprises radiopaque elements arranged in a predetermined pattern, so that a computerized tomography (CT) scanned image of the marker and of the patient enables frames of reference of the patient's anatomy and of the clamp to be registered. (The registration is used in a subsequent stage of the procedure so that images presented to the professional are correctly aligned.)

The CT facility typically comprises an intraoperative CT scanner which has a narrow field of view, so that for the registration to be successful, the radiopaque elements of the marker and of the vertebral bodies should be close. Embodiments of the invention achieve this close proximity by having the marker in the form of a “step,” with one part of the step comprising a plate containing the radiopaque elements, the step-like form of the marker enabling the plate to be positioned close to the vertebral bodies. A second part of the step attaches to the clamp, and there is a known mechanical offset between the two parts.

In embodiments of the present invention the plate is radiotransparent, and has a plurality of radiopaque elements embedded therein in a predefined pattern. To achieve the step-like form described above, a first end of a sigmoid mounting arm is fixedly connected to the radiotransparent plate. In addition, the sigmoid mounting arm has a second end containing one or more fastening receptacles that are configured for removable connection of the arm to the clamp that is attached to the spinous processes.

In a disclosed embodiment the radiotransparent plate, the sigmoid mounting arm, and the surgical clamp are scanned fluoroscopically so as to form a fluoroscopic scan. In response to the fluoroscopic scan and the predetermined mechanical offset, a spatial transformation between the patient and the surgical clamp is determined so as register a frame of reference of the patient with a frame of reference of the surgical clamp.

Typically, in response to registration of the frames of reference, a professional performing surgery on the patient is presented with a stored image of the patient aligned with the patient.

### System Description

In the following, all directional references (e.g., upper, lower, upward, downward, left, right, top, bottom, above, below, vertical, and horizontal) are only used for identification purposes to aid the reader's understanding of the present invention, and do not create limitations, particularly as to the position, orientation, or use of embodiments of the invention.

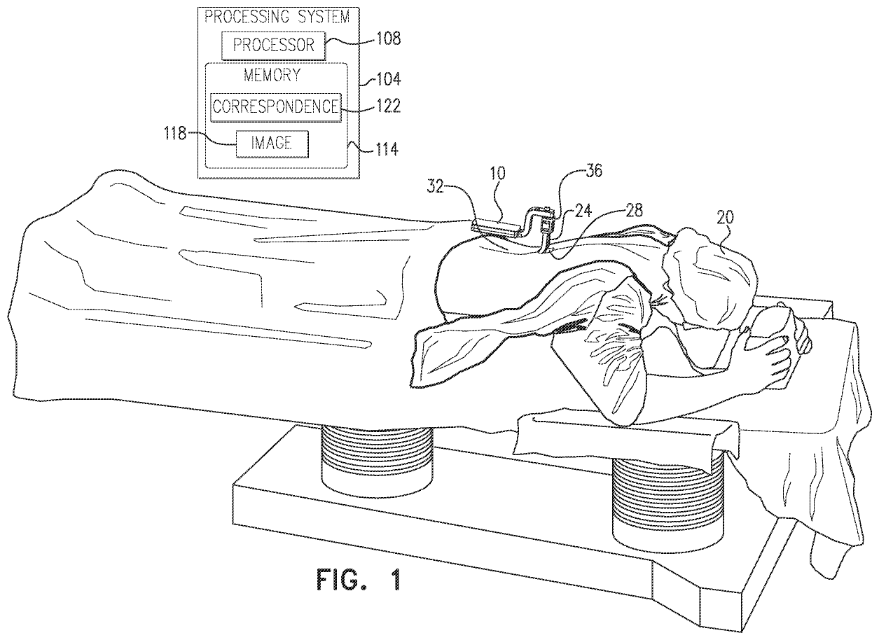

Reference is now made to FIG. 1, which is a schematic diagram illustrating a medical marking device 10, as it is used during a preparatory stage of a medical procedure on a patient 20, according to an embodiment of the present invention. The procedure referred to herein is assumed to comprise a preparatory stage and a subsequent stage, and, as is described in more detail below, device 10 may be used during the preparatory stage. Device 10 is also herein termed a fiducial marker 10. As is also described below, a medical professional uses an augmented reality system during the subsequent stage of the procedure.

The augmented reality system projects virtual images of elements of patient 20 for viewing by the medical professional. The image projection is performed simultaneously with the professional viewing the actual patient, so that the projected images should align with the patient. In order for the projected images in the augmented reality system to align with the patient, frames of reference of the patient and of a clamp attached to a bone of the patient are registered during the preparatory stage, using marking device 10. In the subsequent stage, where the augmented reality system is used, the registration of the clamp computed in the preparatory phase is used to correctly align the images produced by the system.

In the description herein the procedure is assumed to comprise an operation on a spine of patient 20, and in order to perform the registration referred to above, prior to the operation, and in the preparatory stage of the procedure, the medical professional inserts a surgical bone clamp 24 into patient 20. A site of insertion 28 of the clamp is close to, but separate from, a site 32 of the patient's spine to be operated on during the subsequent stage of the procedure. The professional clamps clamp 24 to a section of the spine of the patient, typically to one or more spinous processes of the patient, and the clamp has a support structure 36 to which marker 10 is fastened. A clamp similar to clamp 24 is described in U.S. patent application Ser. No. 16/120, 480 which is incorporated herein by reference.

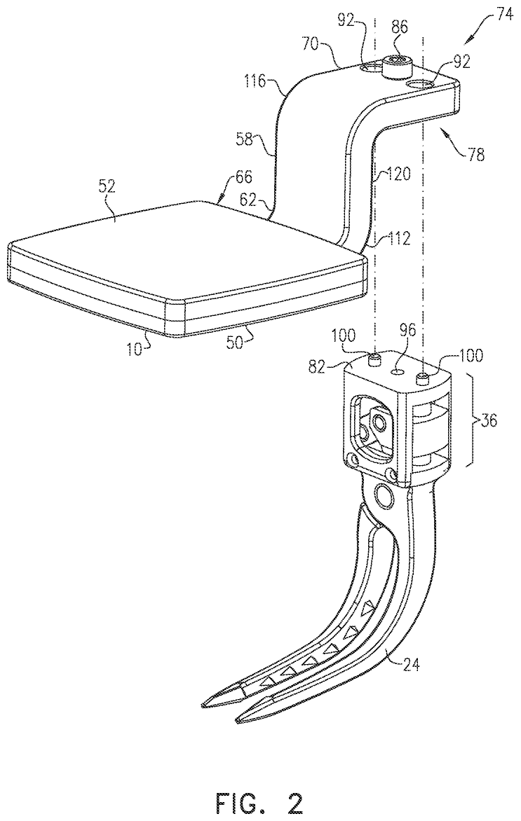

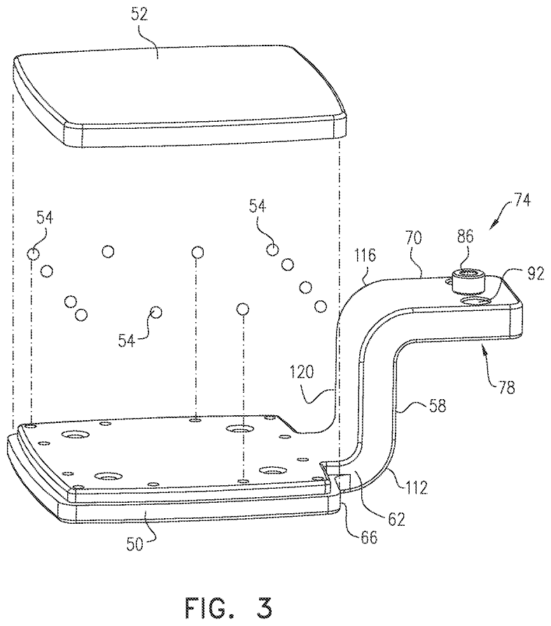

FIG. 2 is a schematic view of device 10 separated from clamp 24, and FIG. 3 is a schematic exploded view of device 10, according to an embodiment of the present invention. Device 10 comprises a radiotransparent plate 50, typically formed of a biocompatible plastic such as a polyimide. A plurality of substantially similar radiopaque elements 54, herein assumed to comprise spheres, are embedded within plate 50. Elements 54 are embedded within plate 50 in a predefined pattern, which is herein assumed to comprise points on sides of a rectangle. However, any other predefined pattern may be used. In some convenient embodiments the predefined pattern has no axis or plane of symmetry. Typically plate 50 is protected by a cover 52.

A radiotransparent sigmoid mounting arm 58, typically formed from the same biocompatible plastic as plate 50, is connected by a first end 62 of the arm to an edge 66 of the plate. A second end 70 of the arm contains one or more fastening receptacles 74 that enable the arm to be securely and fixedly fastened to structure 36 in a predefined orientation. Second end 70 has a lower plane surface 78 which connects with an upper surface 82 of support structure 36. In a disclosed embodiment receptacles 74 comprise a captive screw 86 and holes 92, the screw and the holes mating respectively with a threaded hole 96 and studs 100 in upper surface 82.

As is apparent from the figures, the length of arm 58 determines the separation of plate 50 from lower surface 78 of the arm second end. Embodiments of the present invention comprise sets of devices 10, each member of the set being generally as described herein, but having a different known length of arm 58 from the other set members. Arm 58 is formed from two curved sections 112, 116, separated by a straight section 120, and the different lengths of the arm are formed by varying a length of straight section 120. In embodiments of the present invention the length of straight section 120 varies from 0 to up to approximately 7 cm, although values greater than 7 cm are possible. It will be understood that when the length of section 120 is 0 the two curved sections are connected together with no intervening straight section.

Each member of the set, comprising a respective plate 50 connected to a respective sigmoid mounting arm 58 in a step-like arrangement, may be formed as a single piece, typically by injection molding. It will be understood that each member of the set has known dimensions, so that there is a known mechanical offset between plate 50 and the second end of the arm, including the lower surface 78 of the arm second end.

Typically, for each member of a set of devices 10, the predefined pattern of radiopaque elements 54 is configured to have a one-to-one correspondence with the known mechanical offset. In this case, identification of the pattern provides a unique and unambiguous value for the mechanical offset, and providing the correspondence enables embodiments of the invention to support substantially any offset. As is described below, the correspondence may be stored in a computer memory, and the memory may be accessed so that the unique value of the mechanical offset for a given set member may be determined from the element pattern of the member.

Returning to FIG. 1, device 10 is typically selected from the set of devices so that when the device is attached to upper surface 82, plate 50 of the device is as close as possible to site 32, i.e., to the site of the operation to be performed in the procedure subsequent stage. Once a selected device 10 has been attached to surface 82, a computerized tomography (CT) scan of the device and of the patient's spine is performed. The scan may be performed by inserting patient 20 into a CT scanning facility, typically an intra-operative CT scanner. The insertion may be implemented by bringing the CT scanning facility to patient 20, or by transporting the patient to the facility.

A processing system 104, comprising a computer processor 108 coupled to a memory 114, receives the scan of device 10 and the patient's spine, and stores the scan as an image 118 in the memory. The one-to-one correspondence referred to above may also be stored in memory 114 as a correspondence 122. The processing system is configured to analyze the stored image so as to identify the pattern formed by radiopaque elements 54, and from the identified pattern to register a frame of reference of device 10, and thus of attached clamp 24, with a frame of reference of the patient's anatomy.

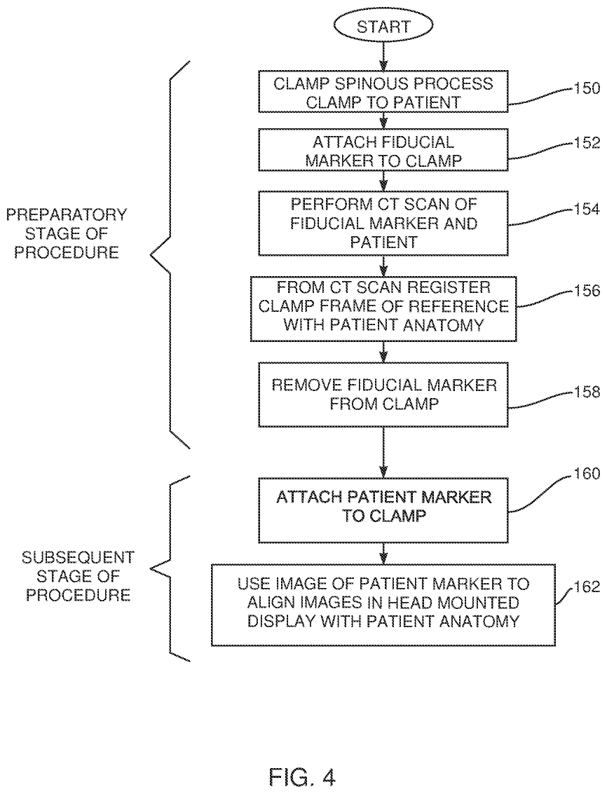

FIG. 4 is a flowchart of steps performed in both the preparatory stage and the subsequent stage of the procedure, according to an embodiment of the present invention. In an initial step 150 and in an attachment step 152 of the preparatory stage, clamp 24 is inserted into patient 20 and clamped to one or more spinous processes of the patient, and fiducial marker 10 is attached to support structure 36 of the clamp, as is described above with reference to FIG. 1.

In a scan step 154 a CT scan of marker 10, attached as described above, is performed, and image 118 of the scan is stored in memory 114. In an analysis step 156 the processing system analyzes the stored image, and from the analysis registers a frame of reference of marker 10, and thus of attached clamp 24, with a frame of reference of the patient's anatomy.

It will be appreciated that the registration of step 156 uses the known mechanical offset of plate 50 with lower surface 78 to provide a location and orientation of upper surface 82 of the clamp with respect to the frame of reference of the patient's anatomy. In the cases where the one-to-one correspondence referred to above is stored as correspondence 122 in memory 114, the processing system may determine the mechanical offset, in analysis step 156, from the pattern identified by the analysis, using the stored correspondence.

Analysis step 156 also determines a spatial transformation between the patient and the surgical clamp, and this is used, together with the mechanical offset, to calculate the registration between the two frames of reference.

In a concluding step 158 of the preparatory stage, fiducial marker 10 is removed from clamp 24, leaving upper surface 82 exposed.

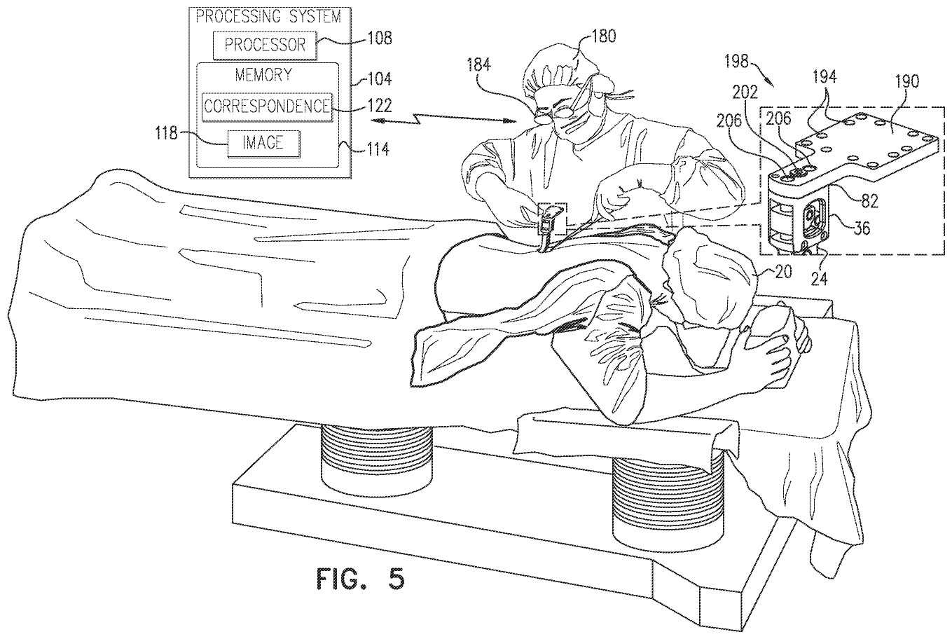

FIG. 5 is a schematic illustration of the subsequent stage of the procedure, according to an embodiment of the present invention. In the subsequent stage a patient marker attachment step 160 and an alignment step 162 are performed, and these steps are described further below. In the subsequent stage, a medical professional 180 operates on the patient. Professional 180 wears an augmented reality head-mounted display (HMD) 184, which is configured to present stored images that are aligned with the patient, to the professional. In order to operate, HMD 184 is coupled to processor 108 of system 104 and the stored images may be stored in memory 114. Alternatively, HMD 184 has its own dedicated processor which performs similar functions to those performed by processor 108. A head-mounted display similar to HMD 184 is described in U.S. Pat. No. 9,928,629, which is incorporated herein by reference.

To perform the alignment for HMD 184, in attachment step 160 a patient marker 190 with known, preset, dimensions, is attached to upper surface 82 of support structure 36 of clamp 24. Marker 190 comprises fastening receptacles 198, substantially similar to receptacles 74, so that in the disclosed embodiment referred to above receptacles 198 comprise a captive screw 202 and holes 206, the screw and the holes mating respectively with threaded hole 96 and studs 100 in upper surface 82 of clamp 24. A patent marker similar to marker 190 is described in PCT Patent Application PCT/IB2019/053524, which is incorporated herein by reference.

Marker 190 comprises optical reflectors 194 incorporated into the surface of the patient marker, and the reflectors are arranged in a predetermined pattern so that an image of the reflectors can be analyzed so as to provide an unambiguous measure of the location and of the orientation of the marker.

In alignment step 162 the HMD projects visible or invisible light to patient marker 190, and acquires images of reflectors 194 of the marker. From the acquired images, the HMD processor determines the location and orientation of the patient marker. Since the patient marker has known dimensions, and is attached to upper surface 82, the processor applies the registration found in step 156 (between the frames of reference of fiducial marker 10 and the patient's anatomy) to ensure that the images projected by the HMD align with the anatomy of patient 20.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1.-15. (canceled)

16. A method, comprising:
attaching a surgical clamp to a bone of a patient;
embedding a plurality of radiopaque elements, arranged in a predefined pattern, in a radiotransparent plate;
connecting a first end of a sigmoid mounting arm to the surgical clamp, the arm having a second end fixedly connected to the radiotransparent plate, and having a predetermined mechanical offset between the first end and the second end;
fluoroscopically scanning the radiotransparent plate, the sigmoid mounting arm, and the surgical clamp so as to form a fluoroscopic scan; and
in response to the predetermined mechanical offset and the fluoroscopic scan, determining a spatial transformation between the patient and the surgical clamp so as register a frame of reference of the patient with a frame of reference of the surgical clamp.

17. The method according to claim 16, wherein the sigmoid mounting arm is radiotransparent.

18. The method according to claim 16, wherein the sigmoid mounting arm comprises a first curved section connected to a second curved section by a straight section.

19. The method according to claim 16, wherein the sigmoid mounting arm comprises a first curved section connected directly to a second curved section.

20. The method according to claim 16, wherein the predefined pattern has no axis of symmetry and no plane of symmetry.

21. The method according to claim 16, and comprising, in response to registration of the frames of reference, presenting to a professional performing surgery on the patient a stored image of the patient aligned with the patient.

22. The method according to claim 16, wherein the predefined pattern and the predetermined mechanical offset are in a one-to-one correspondence.

23. The method according to claim 16, wherein the connecting the first end of the sigmoid mounting arm to the surgical clamp comprises engaging a lower surface of the first end of the sigmoid mounting arm with an upper surface of a support structure of the surgical clamp.

24. The method according to claim 23, wherein the connecting the first end of the sigmoid mounting arm to the surgical clamp further comprises engaging a protrusion extending from one of the upper surface or the lower surface with a hole in the other of the upper surface or the lower surface.

25. A method, comprising:
attaching a support structure to a bone of a patient, the support structure comprising an upper surface;
connecting a first end of a sigmoid mounting arm to the support structure, with a lower surface of the first end of the sigmoid mounting arm engaging the upper surface of the support structure and with at least one of the upper surface or the lower surface comprising a protrusion extending therefrom that engages a hole in the other of the upper surface or the lower surface to align the first end of the sigmoid mounting arm with the support structure, the sigmoid mounting arm having a second end fixedly connected to a radiotransparent plate that comprises a plurality of radiopaque elements embedded therein in a predefined pattern, and wherein the sigmoid mounting arm has a predetermined mechanical offset between the first end and the second end;
fluoroscopically scanning the radiotransparent plate, the sigmoid mounting arm, and the support structure so as to form a fluoroscopic scan; and
causing a processing system to determine a spatial transformation between the patient and the support structure so as register a frame of reference of the patient with a frame of reference of the support structure.

26. The method according to claim 25, wherein the sigmoid mounting arm is radiotransparent.

27. The method according to claim 25, wherein the sigmoid mounting arm comprises a first curved section connected to a second curved section by a straight section.

28. The method according to claim 25, wherein the sigmoid mounting arm comprises a first curved section connected directly to a second curved section.

29. The method according to claim 25, wherein the predefined pattern has no axis of symmetry and no plane of symmetry.

30. The method according to claim 25, and comprising, in response to registration of the frames of reference, presenting to a professional performing surgery on the patient a stored image of the patient aligned with the patient.

31. The method according to claim 25, wherein the predefined pattern and the predetermined mechanical offset are in a one-to-one correspondence.

32. The method according to claim 25, wherein the protrusion comprises a stud extending from the upper surface of the support structure.
